(19) United States
(12) Patent Application Publication
(10) Pub. No.: US 2020/0302445 A1
(43) Pub. Date: Sep. 24, 2020

(54) SYSTEM AND METHOD FOR FACILITATING AND MANAGING SECONDARY TRANSACTIONS RELATED TO HEALTHCARE MARKETPLACES

(71) Applicant: Kalderos, Inc., Chicago, IL (US)

(72) Inventor: Jeremy Docken, Chicago, IL (US)

(73) Assignee: Kalderos, Inc., Chicago, IL (US)

(21) Appl. No.: 16/824,455

(22) Filed: Mar. 19, 2020

Related U.S. Application Data
(60) Provisional application No. 62/820,945, filed on Mar. 20, 2019.

(51) Int. Cl. G06Q 20/40; G06Q 30/02; G16H 20/10

(52) U.S. Cl. CPC G06Q 20/407; G16H 20/10; G06Q 30/0234; G06Q 20/405

(57) ABSTRACT

A method is disclosed for facilitating and managing discounts in the pharmaceutical supply chain, between a discount payer and a discount recipient, the method including the steps of the discount recipient filling a prescription for a consumer; the discount recipient passing a discount request to the discount manager, the discount request seeking a discount payment; the discount manager verifying the discount request from the discount recipient, the discount manager recommending to the discount payer that the discount payment be made by the discount payer to the discount recipient; and the discount payer approving the discount request and remitting discount payment through the discount manager.

### CROSS-REFERENCE TO RELATED APPLICATION

This application claims the benefit of priority to U.S. Provisional Patent Application No. 62/820,945 for a “System and Method for Facilitating and Managing Discounts Relating to the Distribution and Sales of Pharmaceuticals,” filed Mar. 20, 2019, to Docken, which is incorporated by reference in its entirety herein.

### TECHNICAL FIELD

The present invention relates to a system and method for facilitating and managing secondary transactions discounts in healthcare marketplaces. More specific, the present invention relates to a novel chargeback model for discounts relating to the distribution and sales of pharmaceutical product between a drug manufacturer (“Discount Payer”) and a pharmacy, physician office, patient, commercial payer, and/or government agency (“Discount Recipient”), which may be paid directly or indirectly through a novel entity, a “Discount Manager,” in an expeditious manner.

### BACKGROUND

The domestic pharmaceutical supply chain provides for certain systems and methods for distributing pharmaceutical products from a drug manufacturer to a pharmacy or physician office, which are ultimately dispensed to a consumer or patient, and for obtaining payment from the patient and a patient's health insurance plan back through the supply chain to the pharmacy or physician office, drug manufacturer, and various other intermediaries.

Typically in the conventional pharmaceutical supply chain, pharmaceutical product is sold by the drug manufacturer to a wholesaler, which then fulfills replenishment orders at pharmacies or physician offices. As prescriptions are filled, a pharmacy benefits manager working with the pharmacy and a particular patient's health insurance plan attempts to coordinate pricing and reimbursement rates to be sure the pharmacy is ultimately fairly compensated for filling the prescription, the health insurance plan receives discounts or rebates from the drug manufacturer, and the patient pays the appropriate out-of-pocket cost.

The conventional systems and methods involved in the domestic pharmaceutical supply chain for effectuating discounts focus on one or a select handful of preferred pharmaceutical discount programs and do not take into account the entire discount program universe, including the coordination of discounts stemming from or mandated by numerous programs, including but not limited to, federal programs such as the Medicare Prescription Drug Benefit (often referred to as “Medicare Part D” or simply “Part D” coverage), state programs such as the Medicaid Drug Rebate Program (“Medicaid”), commercial managed care formulary discounts, and/or the so-called “340B” drug discounts stemming from Section 340B of the Public Health Service Act of 1992.

None of the conventional systems and methods involved in the domestic pharmaceutical supply chain provide for a system-level solution that provides for the effective coordination of all drug discount programs and/or provides for a point-of-sale discount between the drug manufacturer and the pharmacy, physician's office, and/or patient. Consequently, double-discounting and overpayment/underpayment for drugs is a typical inefficiency in the conventional domestic pharmaceutical supply chain.

The ineffectiveness of conventional systems and methods for preventing double-discounting and overpayment/underpayment for drugs has been a well-researched and documented problem. The Inspector General of the Department of Health and Human Services (“HHS-OIG”) reports unimplemented recommendations to improve federal drug discount program compliance (see, e.g., https://oig.hhs.gov/reports-and-publications/portfolio/drug-pricing/recommendations.asp). The Government Accountability Office (“GAO”) released two reports (January 2020 “Oversight of the Intersection with the Medicaid Drug Rebate Program Needs Improvement” and July 2018 “Improvements Needed in Federal Oversight of Compliance at 340B Contract Pharmacies”) that uncovered additional challenges with the complaint management of drug discount programs. While the federal government continues to perform studies that find failures in conventional systems and methods, little can be done to address the challenges with the current systems and methods due to failures in design.

The method and technology summarized below will fundamentally change how secondary transactions in the healthcare marketplaces such as drug discounts are effectuated today, and will prevent the common and frequent overpayment/underpayment and double-discounting covered in HHS-OIG and GAO reports.

### SUMMARY OF THE INVENTION

According to one non-limiting aspect of the present disclosure, an example embodiment of a method is disclosed for effectuating delivery of pharmaceutical product between a discount payer and a discount recipient, the discount payer and the discount recipient each having a separate computing system having a processor, a memory, and an internet connection, each of the discount payer and discount recipient being further interconnected by a discount manager, the discount manager having a separate computing system having a processor, a memory, and an internet connection. The method includes the steps of the discount recipient procuring the pharmaceutical product at a discounted list price (e.g., wholesale acquisition cost); the discount recipient facilitating the provision of the pharmaceutical product to a patient, the patient having a prescription drug plan administered by a discount stakeholder; and the discount recipient initiating a chargeback request to the discount payer.

According to another non-limiting aspect of the present disclosure, an example embodiment of a method is disclosed for effectuating payment of pharmaceutical discounts in the pharmaceutical supply chain between a discount payer and one or more discount recipients, the discount payer and discount recipient each having a separate computing system having a processor, a memory, and an internet connection, each of the discount payer and discount recipient also being interconnected by a discount manager, the discount manager having a separate computing system having a processor, a memory, and an internet connection. The method includes the steps of the discount recipient facilitating a prescription for a patient; the discount recipient passing a discount request to the discount manager, the discount request seeking a discount payment from a discount payer; the discount manager validating the discount request from the discount recipient, the discount manager recommending to the discount payer that the discount payment be made by the discount payer to the discount recipient; the discount payer approving the discount request and remitting discount payment to the discount manager; and the discount manager relaying payment to the discount recipient.

According to still another non-limiting aspect of the present disclosure, an example embodiment of a method is disclosed for reconciling pharmaceutical discounts paid in the pharmaceutical supply chain between a discount payer and one or more discount recipients, the discount payer and discount recipient each having a separate computing system having a processor, a memory, and an internet connection, each of the discount payer and discount recipient also being interconnected by a discount manager, the discount manager having a separate computing system having a processor, a memory, and an internet connection. The method includes the steps of the discount recipient procuring a pharmaceutical product; the discount recipient facilitating a pharmaceutical prescription for a patient being enrolled in a pharmaceutical plan administrated by a pharmaceutical benefits manager; the discount recipient initiating a pharmaceutical discount request to the discount manager; the discount manager validating the discount request to determine if duplicative discounting exists, and the discount manager confirming the existence of a first and second discount; the discount manager notifying the discount payer of the existence of the first and second discount, the discount payer instructing the discount manager to reprice the pharmaceutical product at the first discount only; the discount manager notifying the discount recipient of the repriced pharmaceutical product; the discount manager receiving payment from the discount payer of the net difference between the first and second discount, the discount manager sending the payment from the discount payer to the discount recipient.

Additional features and advantages are described herein, and will be apparent from the following Detailed Description and the figures.

### BRIEF DESCRIPTION OF THE DRAWINGS

Features and advantages of the system and method for facilitating and managing discounts in the pharmaceutical supply chain described herein may be better understood by reference to the accompanying drawings in which:

FIG. 1 depicts the conventional flow of products and financial transactions among parties in the domestic commercial pharmaceutical supply chain;

FIG. 2 depicts the flow of products and financial transactions between a discount payer and a discount recipient;

FIG. 3 depicts the flow of products and financial transactions between the discount recipient, a discount stakeholder, and a patient;

FIG. 4 depicts a novel discount chargeback model and the exchange of information and financial transactions between the discount payer and the discount recipient;

FIG. 5 depicts the agreement framework between the discount payer, discount manager, and discount recipient that provides for the novel discount chargeback model of FIG. 4;

FIG. 6 depicts an advantage of the novel discount chargeback model of FIG. 4, in which the model intercepts and prevents impermissible double discounting that is typical in the conventional pharmaceutical supply chain of FIG. 1;

FIG. 7 depicts the architecture of an electronic environment of a discount manager facilitating the novel discount chargeback model of FIG. 4;

FIG. 8 depicts the process by which the discount recipient initiates a discount request;

FIG. 9 depicts the process by which the discount manager analyzes a discount request;

FIG. 10 depicts the process by which the discount payer reviews and resolves a discount request; and

FIG. 11 depicts the process by which the discount manager facilitates payment of the discount to the discount recipient.

A skilled artisan will appreciate the foregoing details, as well as others, upon considering the following Detailed Description of certain non-limiting embodiments of the system and method for facilitating and managing discounts in the pharmaceutical supply chain according to the present disclosure. One of ordinary skill also may comprehend certain of such additional details upon using the systems and methods described herein.

### DETAILED DESCRIPTION

The present disclosure, in part, is directed to a system and method for facilitating and managing discounts in the pharmaceutical supply chain. The novel chargeback model disclosed herein facilitates discounts relating to the distribution and sales of pharmaceutical product between a manufacturer or discount payer and a discount recipient, which may be paid directly or indirectly through a discount manager, in an expeditious manner, which has never before existed in the marketplace.

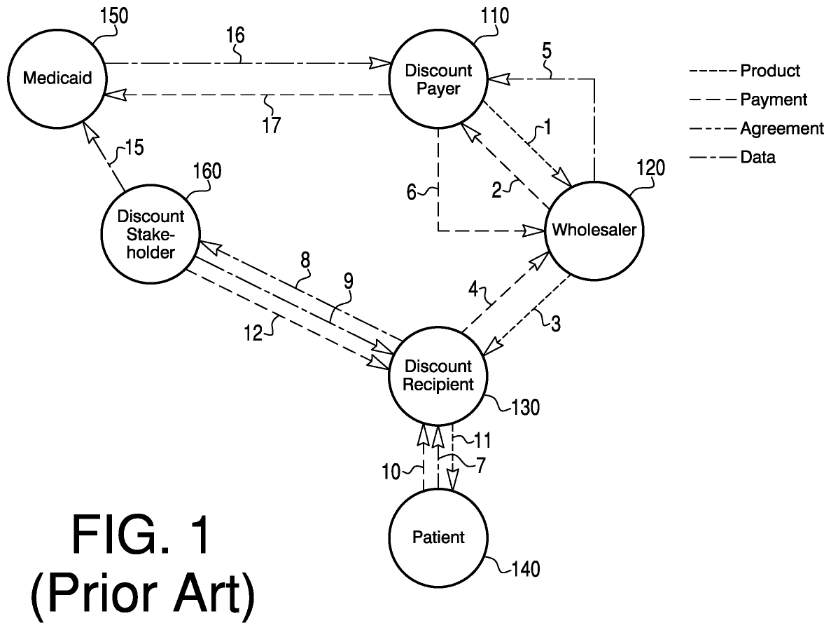

As shown in FIG. 1, a conventional flow of products and financial transactions among parties in the domestic commercial pharmaceutical supply chain is shown. With reference to the numerals shown on FIG. 1, a drug manufacturer or discount payer 110 ships ordered pharmaceutical products, as indicated by reference numeral 1, to a pharmaceutical wholesaler 120. In turn, the wholesaler 120 pays to the discount payer 110 an undiscounted list price, as indicated by reference numeral 2. The wholesaler 120 ships the pharmaceutical product to a pharmacy or discount recipient 130, as shown by reference numeral 3, upon receiving a replenishment order from the discount recipient 130. The discount recipient 130 pays the wholesaler 120 for the product at the price mandated by Section 340B (the “340B price”), as shown by reference numeral 4, and then the wholesaler 120 passes a chargeback request to the discount payer 110 based on the 340B price, as shown by reference numeral 5. As shown by reference numeral 6, the discount payer 110 pays the wholesaler 120 the difference between the undiscounted list price and the 340B price paid by the discount recipient 130 for the pharmaceutical products. One skilled in the art would appreciate that the transaction between the discount payer 110 and the wholesaler 120, as one example, may be made via direct payment or through reconciliation of credit memos.

Typically, at this point, with the pharmaceutical product at the pharmacy or discount recipient 130, the patient 140 provides the discount recipient 130 with the patient's insurance information, as shown by reference numeral 7. The discount recipient 130 will check if the patient 140 is eligible to receive the pharmaceutical product with the discount stakeholder 160, which may comprise a Pharmacy Benefits Manager (“PBM”), as well as communicate the discount recipient's 130 acquisition cost for the pharmaceutical product, as shown by reference numeral 8. The discount stakeholder 160 will send a return message back to the discount recipient 130 authorizing the discount recipient 130 to dispense the product to the consumer or patient 140, the copayment or coinsurance that the discount recipient 130 must collect from the consumer or patient 140, as well as the payment amount the discount stakeholder 160 will reimburse the discount recipient 130 (which is usual and customary to base off of the discount recipient 130 communicated acquisition cost), as shown by reference numeral 9. After collecting the copayment or coinsurance from the consumer or patient 140, as shown by reference numeral 10, the discount recipient 130 dispenses the pharmaceutical product to the consumer or patient, as shown by reference number 11. The discount stakeholder 160 will pay the discount receipt 130 the communicated reimbursement amount, as shown by reference number 12.

With conventional systems, the eligibility of the 340B price is determined post-adjudication, meaning that discounts are applied when product is dispensed to the patient based on assumptions made by the discount recipient and the wholesaler that may be incorrect, leading to duplicate or erroneous discounts paid out by the discount payer 110. For instance, data regarding a pharmaceutical transaction is processed after the product is dispensed to a consumer or patient to evaluate if the initial undiscounted list price transaction met the 340B price eligibility criteria. If eligibility is determined to be correct, then the discount recipient 130 will initiate a “340B replenishment order” with the wholesaler 120 at the 340B price, leading the discount payer 110 to pay a 340B chargeback to the wholesaler 120. The discount recipient 130 is usually required to update the acquisition cost originally communicated, as shown by reference number 8, to indicate that the acquisition cost was a discounted 340B price, but this process is prone to error, or simply does not occur. As a result, the discount stakeholder 160 often requests a commercial discount from the manufacturer, as shown by reference number 13, not knowing that the transaction had been filled with a product sold subject to a 340B discount (knowledge of which should cause the discount stakeholder 160 to not request a commercial discount). Consequently, the discount payer 110 frequently pays an erroneous duplicate discount, as shown by reference number 14.

In this particular scenario, as will become apparent in the conventional reimbursement process described herein, if the patient was covered by a state Medicaid plan 150, the discount stakeholder 160 will submit a summary of reimbursement transactions made during the quarter, as shown by reference numeral 15. Included in this summary is information regarding which reimbursements were made based on an undiscounted list price acquisition cost, and which reimbursements were made based on a 340B priced acquisition cost. Because the original acquisition cost communicated by the discount recipient 130 to the discount stakeholder 160, shown by reference numeral 8, is frequently incorrect, the state Medicaid program office 150 frequently submits an erroneous request for a Medicaid discount to the discount payer 110, as shown by reference numeral 16. In the conventional pharmaceutical supply chain, there is no effective mechanism in place for the discount payer 110 to know that the Medicaid discount requested by the state Medicaid program office 150 is requested on a transaction which the discount payer 110 already paid a 340B discount to the discount recipient 130. Consequently, the discount payer 110 frequently pays an erroneous duplicative discount to the state Medicaid program office 150, as shown by reference numeral 17.

The novel chargeback model described herein involves the inclusion of a discount manager 170 to oversee the entire discounting universe. The inclusion of a discount manager 170 provides for many advantages described herein and which will become apparent to one of ordinary skill based on the following description in view of the accompanying drawings.

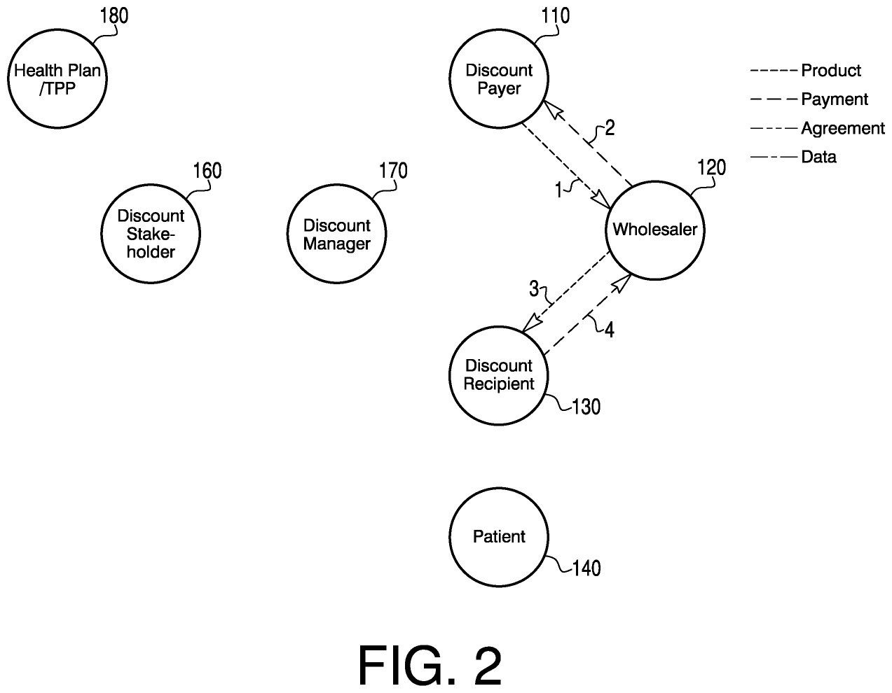

As shown in FIG. 2, the discount recipient 130 acquires pharmaceutical product from the discount payer 110 in much the same way as currently exists in the marketplace, whereby a wholesaler 120 purchases product from the discount payer 110 at an undiscounted list price. One important distinction in the acquisition process, however, is that the discount recipient 130 also purchases pharmaceutical product from the wholesaler 120 at the undiscounted list price, as shown by reference numeral 3, thereby eliminating the need for the wholesaler 120 to seek a chargeback request from the discount payer 110. This sets up an important advantage by the novel chargeback model described herein, in which the discount recipient 130 may directly request a chargeback from the discount payer 110 at the point-of-sale.

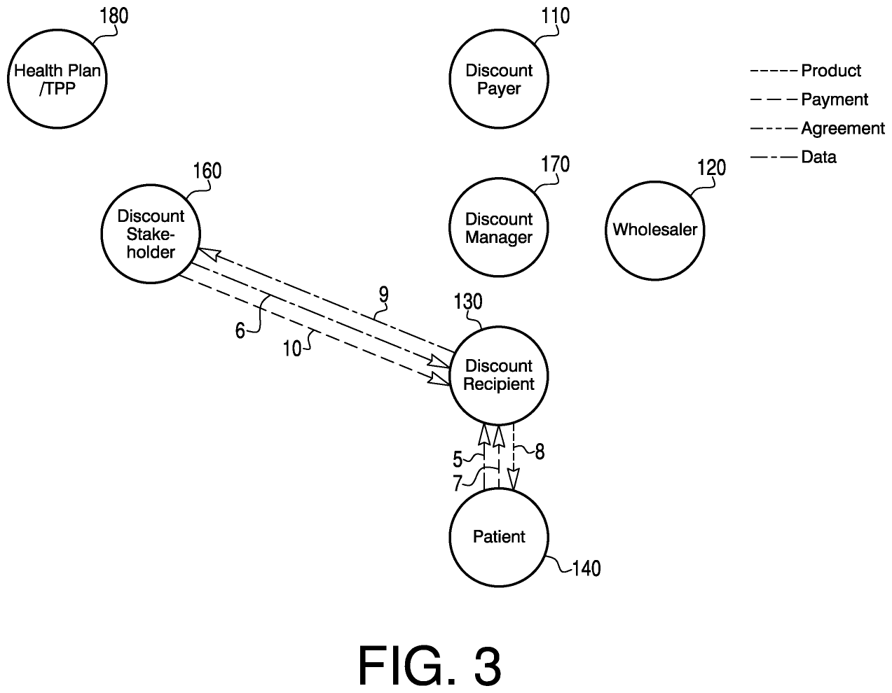

As shown in FIG. 3, the novel chargeback model described herein provides efficiencies in the dispensing of pharmaceutical product to the patient 140. The process begins at reference numeral 5 with the patient 140 presenting his or her insurance card to the discount recipient 130. The discount recipient 130 runs the insurance card through a switch interconnected with the discount stakeholder 160 at reference numeral 9, which approves the transaction at reference numeral 6 and returns to the discount recipient 130 the patient's fixed contract price and co-pay or co-insurance amount. The patient 140 pays the co-pay or co-insurance at reference numeral 7, and the discount recipient 130 provides the pharmaceutical product to the patient at reference numeral 8. The discount stakeholder 160, in turn, reimburses the discount recipient 130 the acquisition price of the pharmaceutical product at reference numeral 10.

The patient's 140 healthcare plan 180 may be a state Medicaid program 150, or may be any number of public, private, or governmental-sponsored programs, and the novel chargeback model described herein is configured to work with any discounting program, including but not limited to: 340B Drug Discount Programs, Veterans Affairs Pricing/Federal Supply Schedule, Wholesaler Sourcing, GPOs, Medicaid Drug Rebate Program, Commercial Managed Care Rebates, Medicare Part D Rebates, Medicare Coverage Gap, co-pay coupon cards, Patient Assistance Programs, Other Government Discounts (e.g., TRICARE, State Pharmaceutical Assistance Programs), and any other healthcare or discount programs that may come into existence in the future.

The interplay between the discount recipient 130 and the discount stakeholder 160 varies from one patient 140 to another, and is dependent, in part, upon the patient's healthcare plan 180 and the extent to which the patient may be enrolled in a government program, such as Medicaid or Medicare, and/or a private healthcare plan as primary healthcare or as a supplement to a government plan. In some instances, the discount recipient 130 may be the discount stakeholder 160, which submits a discount request to the discount payer 110. In instances where the discount recipient 130 is the PBM, the discounts often take the form of a rebate, which may or may not involve the transaction of payment but rather may be in the form of remittance, credit note, or some other change in the ledger between the parties. In other instances, the discount recipient 130 may be the consumer or patient 140, who may receive discounts from discount payer 110, discount stakeholder 160, health plan/TPP 180, and/or discount manager 170, which may be used towards healthcare services. In instances where the discount recipient 130 is the consumer or patient 140, the discounts may take the form of a deposit to a prepaid card or a health savings account. The complexity and number of relationships that may exist between the discount recipient 130 and the discount payer 110, including the presence of any discount stakeholder 160 separate from the discount recipient, are reasons why the state of the discounting universe is so uncertain and inefficient, and these reasons give rise to the need in the art for the novel chargeback model disclosed herein and facilitated by the discount manager 170 described in the present invention.

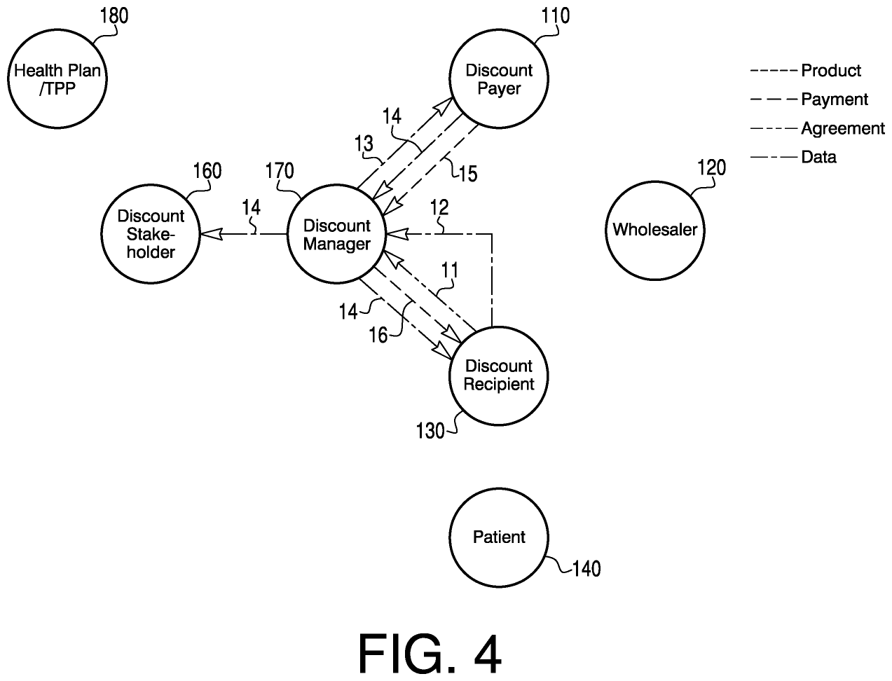

As shown in FIG. 4, the essence of the novel chargeback model described herein is facilitated by the discount manager 170. Participating discount recipients 130 enter into an agreement with the discount manager 170, as shown by reference numeral 11, similar to the agreements that a discount recipient 130 may enter into with a PBM or discount stakeholder 160.

The chargeback process is initiated when the discount recipient 130 submits a request for chargeback to the discount manager 170, as shown by reference numeral 12. The chargeback is calculated as the difference between the undiscounted list price and a discounted price. The discount manager 170 validates the chargeback request and provides a recommendation to the discount payer 110, as shown by reference numeral 13, to pay or fail the discount request in accordance with predefined rules set by the discount payer 110. The predefined rules may be self-imposed on the discount payer 110 or may result from applicable laws or contractual agreement entered into by the discount payer 110. The discount payer 110 resolves the discount request and notifies the discount manager 170, which relays the decision to the discount stakeholder 160 and the discount recipient 130, as shown by reference numeral 14. To the extent that the discount request is approved, the discount manager 170 facilitates the transfer of payment from the discount payer 110 to the discount recipient 130, as shown by reference numeral 16. In this arrangement, chargebacks flow directly from the drug manufacturer or discount payer 110 to the discount recipient 130.

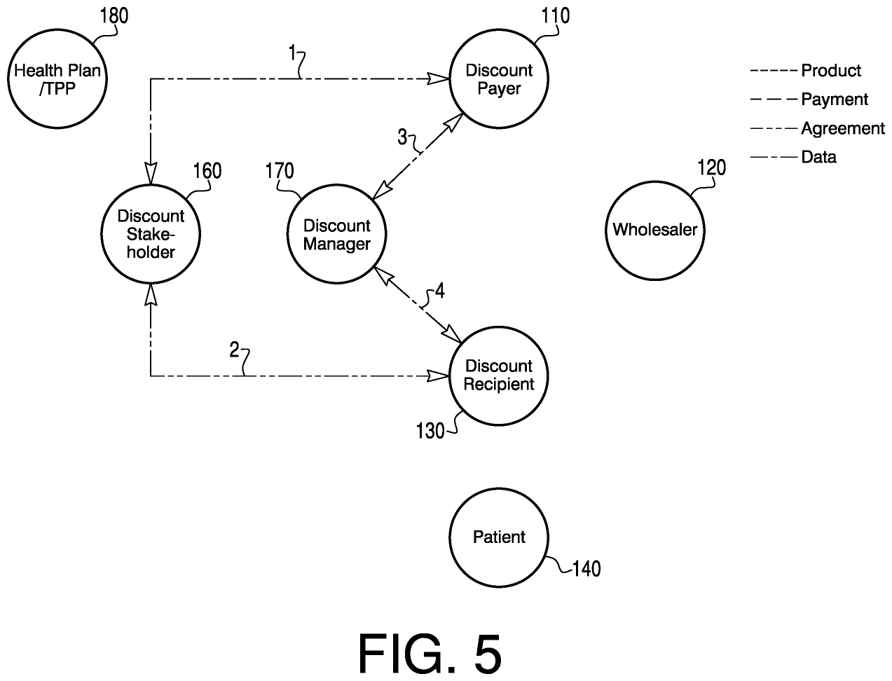

As shown in FIG. 5, an agreement framework is necessary to facilitate the novel chargeback model described herein, and to be sure that a dispensed product subject to a 340B price discount is not duplicatively discounted. As shown by reference numeral 1 in FIG. 5, the discount payer's 110 contract with the discount stakeholder 160 excludes contract pricing if the dispensed drug is subject to a 340B price discount. As shown by reference numeral 2, the discount stakeholder's 160 contract with the discount recipient 130 honors the discount payer's 110 contract exclusions relating to 340B price discounts. As shown by reference numeral 3, the discount payer's 110 contract with the discount manager 170 requires that the discount manager abide by the discount payer's 110 decision regarding contract and discount is applicable to a discount-eligible transaction. As shown by reference numeral 4, the discount manager's 170 agreement with the discount recipient 130 provides that a previously-paid discount may be reversed prior to new contract pricing being applied to the transaction if the discount recipient 130 attempts to re-price a previously-processed transaction.

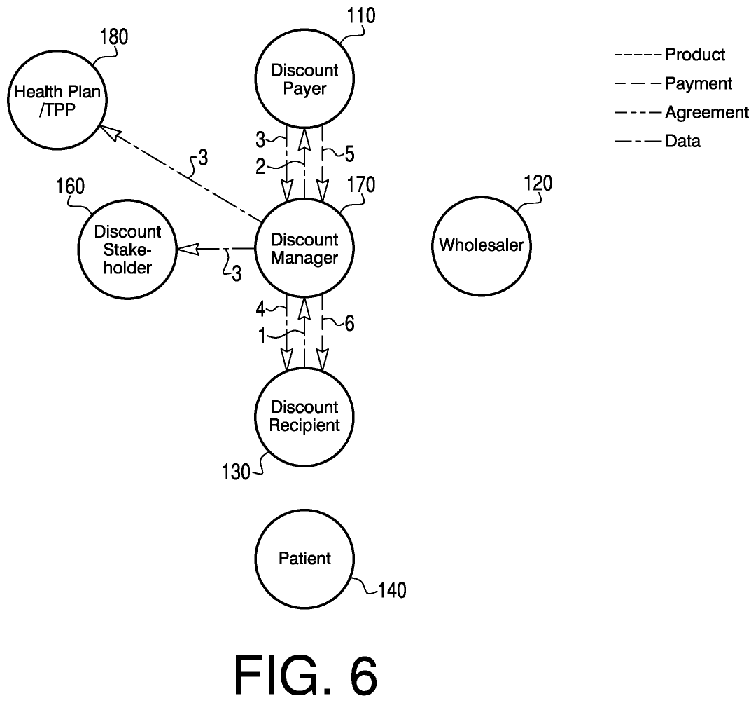

An advantage of the novel chargeback model described herein is the proper policing and oversight of 340B price discounts required by federal law. As shown in FIG. 6, a discount recipient 130 or other covered entity may retroactively classify a script as being eligible for 340B pricing, at which point a 340B replenishment chargeback request is made by the discount recipient 130 to the discount manager 170, as shown by reference numeral 1. The discount manager 170 validates the request to determine if duplicate discounting (both 340B and PBM discounting) were applied, and if so, discount manager 170 notifies the discount payer 110, as shown by reference numeral 2. Because the 340B price discount is mandated by law, the discount payer 110 instructs the discount manager 170 to reverse or dispute any previously-paid discounts that have been requested by or paid to the discount stakeholder 160 and/or the health plan/TPP 180, as shown by reference numeral 3. The discount manager 170 notifies the discount recipient 130, as shown by reference numeral 4, that prior discounts paid to the discount stakeholder 160 and/or the health plan/TPP 180 have been reversed or disputed, and that the discount recipient 130 will be paid the requested 340B replenishment chargeback. The discount manager 170 facilitates payment of the net discount difference between the undiscounted list price and the 340B price, and payment is made from the discount payer 110 to the discount recipient 130, as shown by reference numeral 5.

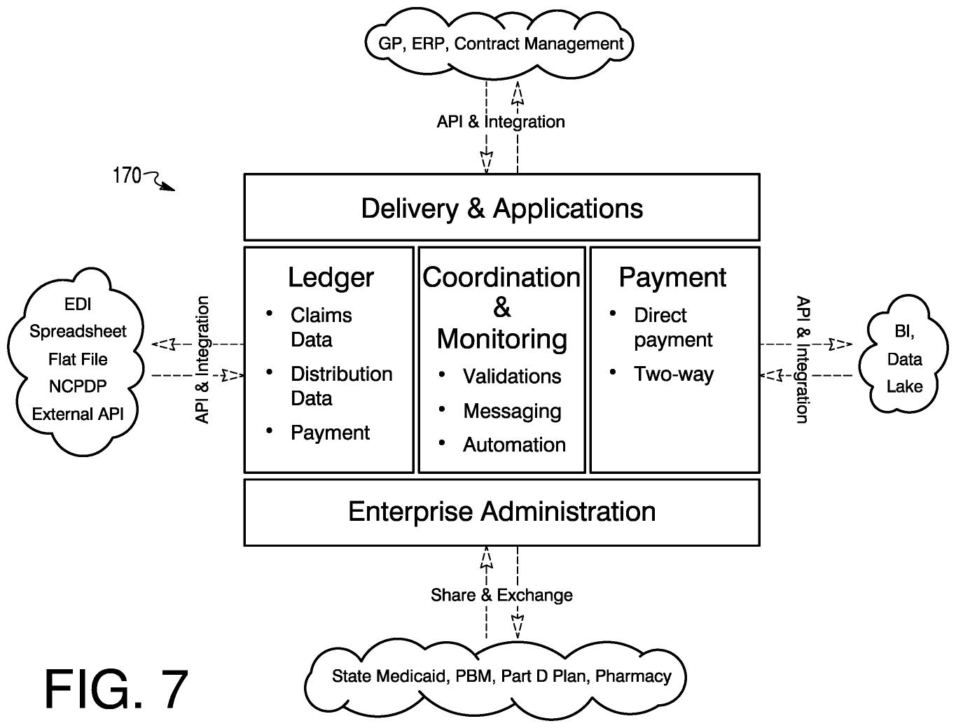

To facilitate the functionality of the discount manager 170, which is essential to carrying out the novel chargeback model described herein, an electronic architecture is provided to bring the discount payer 110, discount recipient 130, discount stakeholder 160, and healthcare plan 180, which may include a state Medicaid program office 150, together. FIG. 7 shows the connectivity of one such electronic architecture.

As shown in FIG. 7, discount manager 170 provides at least three categories of data analysis and transformation, including: (1) serving as a ledger between the parties, for claims data, for distribution data, and for payment and financial transactions; (2) providing coordination and monitoring, for validations of discount chargebacks and rebate requests, messaging between parties as to the status of a claim, and automation of claims and payments among the parties; and (3) facilitating payment between the parties, particularly the discount payer 110 and the discount recipient 130, including facilitation of direct payment and two-way payment.

Consistent with what is shown in FIG. 7, the discount manager 170 provides this functionality by serving as both an exchange between the parties and also by serving as a central repository of data to allow the parties to each have all of the information necessary to carry out respective tasks. First, discount manager 170 serves as an exchange between the discount recipient 130 and healthcare plans 180, including Medicaid 150, to provide for the sharing of pricing and discount or rebate information relating to the filling of each script. In this manner, potentially duplicative discounts can be intercepted before they are paid out, or chargebacks can be reversed, in part or in whole, to correct the amount ultimately paid out by the discount payer 110. In this manner, discount manager 170 serves as an enterprise administrator for the entire discount universe, which does not exist in the current marketplace.

Second, discount manager 170 serves as a central repository and interface for many other aspects of the pharmaceutical supply chain, including accounting, enterprise resource planning (“ERP”), contract management, business intelligence (“BI”), and other functions and applications. This delivery of information can be coordinated and carried out by offering ERP and BI solutions certain access via application programming interface (“API”) endpoints, electronic data interchange (“EDI”) connections, or other types of integration with the discount manager's database. For example, the discount manager 170 may send or receive information to other parties in the pharmaceutical supply chain using an API or EDI, external API, or through a flat file, spreadsheet, or other file or data type. Information managed by the discount manager 170 may be coded with identifiers from the National Council for Prescription Drug Programs (“NCPDP”) with respect to discount payers 110, discount recipients 130, and/or scripts. The discount manager 170 may serve as a data lake from which BI and ERP applications access, gather, and compile information.

The database or central repository of discount manager 170 is carried out on one or more servers, which may be cloud-based, interconnected with one another and with the world wide web and/or other network connections. An exemplary computing environment upon which embodiments of the present invention may be implemented includes a general-purpose computing system environment, such as computing system. In its most basic configuration, a computing system typically includes at least one processing unit and memory, and an address/data bus (or other interface) for communicating information. The computing system environment may be part of a cloud-based computing environment. For example, a storage host, a web server and an application engine may individually or collectively be implemented as or hosted on one or more such computing system environments.

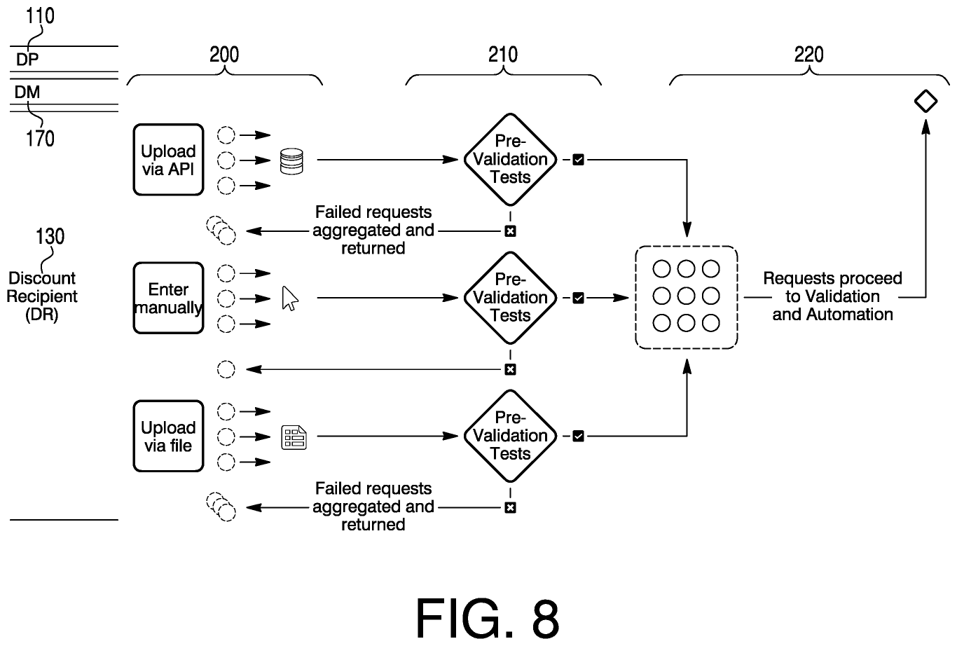

An example of the specific functionality that discount manager 170 may employ, and its interconnectivity with the discount payer 110 and the discount recipient 130 is described and depicted in FIGS. 8-11. As shown in FIG. 8, the discount manager 170 provides a centralized process for discounts to be requested from the discount recipient 130. Preferably, discount recipient 130 uploads the discount request immediately via an API as shown at 200, although discount requests may be uploaded via a file or other format or may be entered manually. An initial pre-validation is conducted on the discount requests, as shown at 210, to be sure that the requests are facially complete, and pre-validated requests are passed at 220 to the discount manager 170 for analysis.

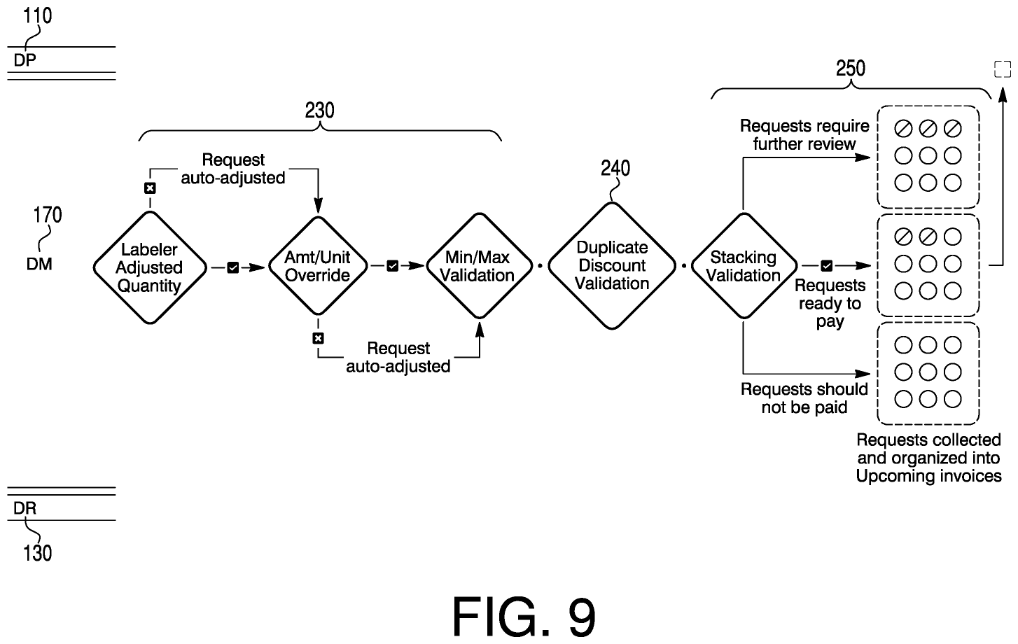

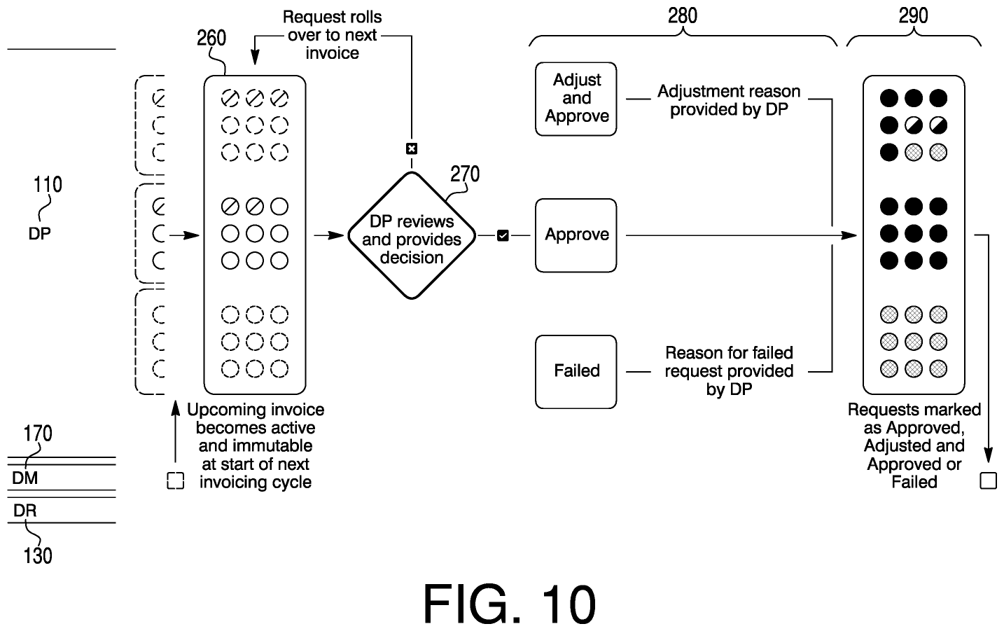

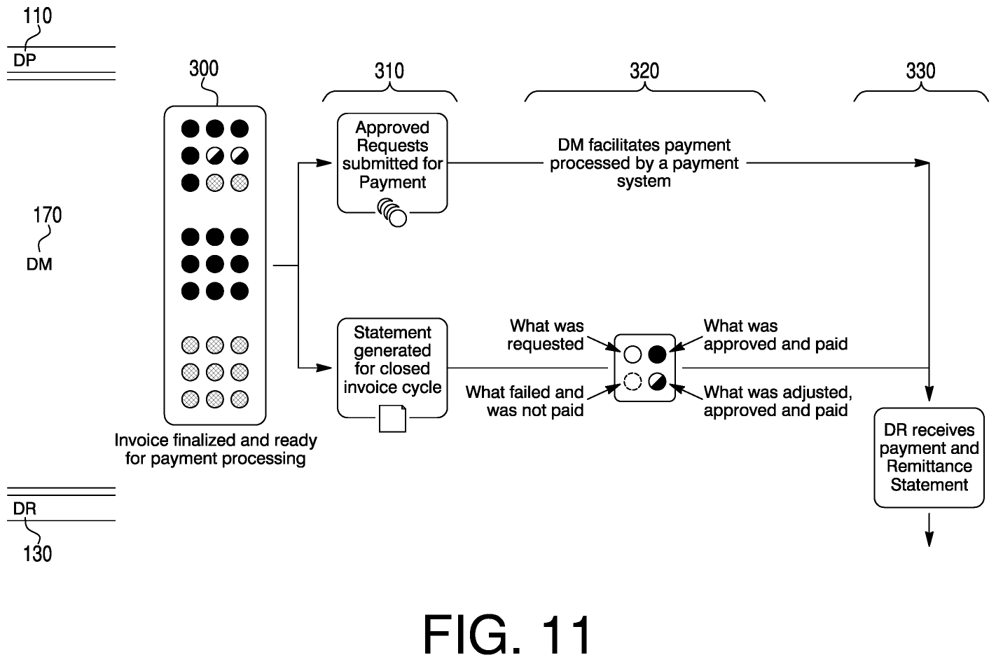

As shown in FIG. 9, discount manager 170 carries out steps at 230 to validate the discount requests from discount recipient 130, including detecting, at 240, whether a duplicate discount already exists in the pricing charged or has been paid out by the discount payer 110. The discount manager 170 returns at 250 a stacking validation, identifying those requests that should be paid, those that should not, and those that may require further review. With respect to those discount requests flagged for further review, as shown in FIG. 10, those requests are rolled to the next invoice at 260, and discount payer 110 reviews the requests and makes a determination. Discount payer 110 may determine at 280 to take no action and allow the discount to be paid, may adjust the request and approve, or may fail the discount request to the extent that a discount or rebate already was applied. The discount payer's results are passed at 290 back to the discount manager 170 for final disposition. As shown in FIG. 11, the discount manager 170 prepares the final invoice for the discount recipient 130 at 300 taking in the final determinations of all the discount requests. Approved requests are forwarded at 310 for payment and a statement is generated for the closed invoicing cycle. At 320, discount manager 170 may conduct payment of the discounts directly, such as by using an electronic payment service or similar ACH processing or transactions, although payment also may be made in the form of a credit memo or other offset. At 330, the discount recipient 170 receives a remittance statement detailing the entire discounting transaction.

The novel chargeback model described herein provides for numerous advantages, some non-limiting examples of which are detailed as follows. As to the pharmacy or discount recipient 130, one advantage is that discounts may be provided as direct cash payments (or credit memos), from the discount payer 110 directly or through the discount manager 170, rather than as credits toward future drug purchases from the wholesaler 120. The seamless integration with the discount manager 170 allows for the chargeback model to correct errors in previously adjudicated discounts by the discount payer 110 or recipient 130 without requiring the discount recipient 130 to credit and rebill the original transaction. Moreover, it allows the chargeback model to correct beneficiary overpayment should errors be discovered after a script is dispensed to a patient 140.

Another advantage of the novel chargeback model for the discount recipient 130 is that implementation does not require the pharmacy or discount recipient 130 to switch to particular wholesalers or vendors in order to receive discounts, which is a departure from the conventional supply chain where PBMs often dictate to pharmacies which wholesalers they use. Much to the contrary, implementation of the novel chargeback model described herein is flexible. Any pharmacy management software provider can connect directly to an API endpoint of the discount manager 170, or the pharmacy or discount recipient's 130 switch can route discount requests and responses to the API endpoint.

Still another advantage of the novel chargeback model to discount recipients 130 is that embedded validations through the discount manager 170 reduces time between submission of data from the discount recipient 130 and the response from the discount payer 110 with respect to approval of discount requests. These efficiencies bring down costs for all parties, potentially improve and simplify working capital management for discount recipient 130, expedite the script filling and billing processes, and generally improve the consumer or patient experience.

As to the PBM or discount stakeholder 160, the novel chargeback model described herein provides for numerous advantages, some non-limiting examples of which are detailed as follows. One advantage is that the status of manufacturer or discount payer's 110 discount payment to the pharmacy or discount recipient 130 is shared with the discount stakeholder 160. The discount stakeholder 160 may be integrated directly with the discount manager 160 via an API endpoint or similar integration or interconnection. As such, the discount stakeholder 160 can better manage its payment of discounts, knowing which discounts have been approved by the discount payer 110, and at what time.

Another advantage of the novel chargeback model to the discount stakeholder 160 is that it reduces the potential for disputes between the discount payer 110 and the discount stakeholder 160, as the discount payer 110 will be able to validate and approve claims before any discount is approved by the discount stakeholder 160. If disputes should occur, the novel chargeback model provides for a common dispute resolution scheme to be applied across all drug manufacturers or discount payers 110.

As to the drug manufacturer or discount payer 110, the novel chargeback model described herein provides for numerous advantages, some non-limiting examples of which are detailed as follows. One advantage is that it provides the discount payer 110 the ability to validate and approve any discount before it is paid. Similarly, the point-of-sale chargeback model provides the discount payer 110 the ability to ensure discount requests are in compliance with federal and state laws and contract terms. These advantages save the discount payer 110 and the entire supply chain money, which ultimately may bring down the cost of prescription drugs to the patient 140. These advantages additionally help manage or reduce potential liability risk of the discount payer 110 by more effectively managing or limiting non-compliance exposure.

Another advantage of the chargeback model is that it allows the discount payer 110 to set up one payee in an ERP system, that being the discount manager 170. In the conventional pharmaceutical supply chain, drug manufacturers remit discounts to thousands or tens of thousands of parties (PBMs, pharmacies, etc.), and each one is a separate ERP payee, thereby triggering separate accounts payable and separate tax reporting requirements. By dealing with one discount manager 170, the discount payer 110 foregoes having to deal with tens of thousands of payees, accounts payables, and tax reporting requirements and instead can dedicate more time and resources to its core business of developing life-saving drugs.

This advantage saves the discount payer 110 in the near term, which has a significant savings impact on the entire supply chain. For example, it creates a single source of truth for all drug discount requests and payment status. This advantage has the impact of increasing efficiencies in the pharmaceutical supply chain, thereby potentially bringing prescription drug prices down.

As to the consumer or patient 140, the novel chargeback solution proves a mechanism that allows multiple different parties, including but not limited to drug manufacturers, device manufacturers, health insurers, PBMs, hospitals, clinics, government agencies, and not-for-profit entities to channel discount dollars to a prepaid card or health savings account connected to the discount manager 170 and use these discount dollars towards out-of-pocket healthcare costs.

This advantage allows private entities and policy makers to direct some or all of the billions of dollars of discount given by discount payers 110 to business in the supply chain, which unfortunately are not often shared with the consumer or patient 140, a mechanism to direct those discounts to helping individuals pay for healthcare products and services. This advantage has the impact of helping millions of people afford the ever-increasing cost of healthcare.

As an enterprise solution, the novel chargeback model described herein enables effective coordination of all programs rather than looking at each discount program as a stand-alone program. It is a system-level solution that considers the entire drug discount program universe. As such, the novel chargeback model described herein can effectuate a point-of-sale discount between the drug manufacturer or discount payer 110 and the pharmacy or discount recipient 130, which does not take place in the conventional pharmaceutical supply chain.

While the novel point-of-sale chargeback model described herein has been described in detail with respect to the distribution and sales of pharmaceutical product, the novel chargeback model is uniquely suited to work with other secondary transactions related to healthcare marketplaces. For instance, the unique chargeback model also is entirely applicable with the distribution and sales of medical devices. Examples of medical devices include, but are not limited to: hospital beds, lifts, commode chairs; infusion pumps and supplies; blood glucose monitors and test strips; canes, crutches, walkers, and wheelchairs; oxygen, nebulizers, and nebulizer supplies and medications; continuous positive airway pressure machines and supplies; implantable medical devices such as pacemakers and defibrillators; and any other medical devices covered under healthcare or discount programs.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended set of claims.

What is claimed is:

1. A method for effectuating delivery of pharmaceutical product between a discount payer and a discount recipient, the discount payer and the discount recipient each having a separate computing system having a processor, a memory, and an internet connection, each of said discount payer and discount recipient being further interconnected by a discount manager, said discount manager having a separate computing system having a processor, a memory, and an internet connection, the method comprising the steps of:
said discount recipient procuring said pharmaceutical product at an undiscounted list price;
said discount recipient facilitating the provision of said pharmaceutical product to a patient, said patient having a prescription drug plan administered by a discount stakeholder; and
said discount recipient initiating a chargeback request to said discount payer.

2. The method of claim 1 wherein the discount stakeholder is the discount recipient and initiates said chargeback request by submitting a request for chargeback to said discount manager.

3. The method of claim 12 wherein the discount manager determines the chargeback amount as being the difference between the undiscounted list price and the total amount collected by said discount recipient from said discount stakeholder, less any payment made by said patient to said discount recipient.

4. The method of claim 3 wherein the discount manager validates the chargeback request based on a set of rules defined by said discount payer based on federal and state laws and any contractual agreements binding said discount payer.

5. The method of claim 4 wherein the discount payer resolves the chargeback request and notifies said discount manager of the decision, the discount manager notifying the discount recipient and/or the discount stakeholder of said decision.

6. The method of claim 5 wherein the discount payer approves said chargeback request, said discount payer sending payment directly to said discount recipient.

7. A method for effectuating payment of pharmaceutical discounts between a discount payer and a discount recipient, the discount payer and the discount recipient each having a separate computing system having a processor, a memory, and an internet connection, each of said discount payer and discount recipient also being interconnected by a discount manager, said discount manager having a separate computing system having a processor, a memory, and an internet connection, the method comprising the steps of:
said discount recipient facilitating a prescription for a patient;
said discount recipient passing a discount request to the discount manager, said discount request seeking a discount payment from a discount payer;
said discount manager validating said discount request from said discount recipient, said discount manager recommending to said discount payer that said discount payment be made by said discount payer to said discount recipient;
said discount payer approving said discount request and remitting discount payment to said discount recipient; and
said discount manager facilitating payment from said discount payer to said discount recipient.

8. The method of claim 7 wherein said discount recipient initiates said discount request by transmitting said request via an application programming interface or user interface.

9. The method of claim 7 wherein said discount recipient requests a plurality of discount requests relating to more than one prescription having been filled.

10. The method of claim 9 wherein said discount manager reviews said plurality of requests and returns to said discount payer a set of requests to pay, a set of requests not to pay, and a set of requests for review by said discount payer.

11. The method of claim 10 wherein said discount payer resolves said plurality of requests and initiates payment to said discount recipient of approved discounts.

12. A method for reconciling the payment of pharmaceutical discounts between a discount payer and a discount recipient, the discount payer and the discount recipient each having a separate computing system having a processor, a memory, and an internet connection, each of said discount payer and discount recipient also being interconnected by a discount manager, said discount manager having a separate computing system having a processor, a memory, and an internet connection, the method comprising the steps of:
said discount recipient procuring a pharmaceutical product;
said discount recipient facilitating a pharmaceutical prescription for a patient, said patient being enrolled in a pharmaceutical plan administrated by a pharmaceutical benefits manager or public or private health insurer;
said discount recipient initiating a pharmaceutical discount request to said discount manager;
said discount manager validating said discount request to determine if duplicative discounting exists, and said discount manager confirming the existence of a first and second discount;
said discount manager notifying said discount payer of the existence of said first and second discount, said discount payer instructing said discount manager to reprice said pharmaceutical product at said first discount only;
said discount manager notifying said discount recipient of said repriced pharmaceutical product;
said discount manager receiving payment from said discount payer of the net difference between said first and second discount, said discount manager sending said payment from said discount payer to said discount recipient.

13. The method of claim 12 wherein the discount manager determines said second discount to be duplicative and reverses said second discount.

14. The method of claim 13 wherein the discount manager reprices said pharmaceutical product to be in compliance with said first discount.

15. The method of claim 14 wherein the discount payer pays said discount recipient the net difference between said first and second discount.

16. The method of claim 14 wherein the discount payer pays said discount manager the net difference between said first and second discount.

17. The method of claim 12 wherein the first discount is a Rule 340B discounted price and said second discount is a discounted price in compliance with said pharmaceutical benefits manager.

18. The method of claim 17 wherein the discount manager reverses said second discount applied by said pharmaceutical benefits manager.

19. The method of claim 18 wherein the discount manager reprices said pharmaceutical product to be said Rule 340B discounted price.

20. The method of claim 19 wherein the discount payer pays said discount recipient the net difference between said second discount applied by said pharmaceutical benefits manager and said Rule 340B discounted price.

21. The method of claim 19 wherein the reversed second discount is either paid directly from the pharmaceutical benefits manager to the discount payer or held as a credit and used towards the payment of future discount requests placed by the pharmaceutical benefits manager to the discount payer.
